(19) United States
(12) Patent Application Publication
(10) Pub. No.: US 2005/0113365 A1
(43) Pub. Date: May 26, 2005

(54) Method and medicine for treating gastrointestinal disorder including irritable bowel syndrome

(75) Inventor: James E. Lundeen, Cleveland Heights, OH (US)

Correspondence Address: Warren A. Sklar;Renner, Otto, Boiselle & Sklar, LLP, Nineteenth Floor, 1621 Euclid Avenue, Cleveland, OH, 44115-2191, US

(73) Assignee: SIR ISAAC NEWTON ENTERPRISES LLC, Cleveland Heights, OH (US)

(21) Appl. No.: 10/970,164

(22) Filed: Oct. 21, 2004

Related U.S. Application Data
(60) Provisional application No. 60/518,715, filed on Nov. 10, 2003.
(60) Provisional application No. 60/518,718, filed on Nov. 10, 2003.
(60) Provisional application No. 60/518,719, filed on Nov. 10, 2003.

(51) Int. Cl. A61K 31/551; A61K 31/5415

(52) U.S. Cl. 514/220; 514/224.8; 514/250

(57) ABSTRACT

A method and a medicine for treating a human having a gastrointestinal disorder that includes irritable bowel syndrome are provided. The method includes administering a dose of the medicine to the human. The medicine includes a tricyclic antidepressant and a stool softener.

### CROSS-REFERENCE TO RELATED APPLICATIONS

This application claims priority to U.S. Provisional Patent Application Ser. No. 60/518,715 filed on Nov. 10, 2003; 60/518,718 filed on Nov. 10, 2003; and 60/518,719 filed on Nov. 10, 2003, the disclosures of which are hereby incorporated by reference in their entirety.

### BACKGROUND

1. Field of Invention

The present invention generally relates to a method and a medicine for reducing or eliminating the undesirable affects of a gastrointestinal disorder. More particularly, the invention relates to a method for reducing or eliminating symptoms of irritable bowel syndrome. The invention also relates to a medicine for reducing or eliminating symptoms of irritable bowel syndrome.

2. Discussion of Related Art

Irritable bowel syndrome (IBS) may be either an acute or a chronic functional disorder of the lower bowel and is believed to affect between ⅕ and ¼ of all adults. An acute attack may last only a few days or weeks, and is not recurring over a long period of time. In contrast, a chronic affliction may last or persist for a long time, or may reoccur regularly or irregularly over a long time.

A manifestation of irritable bowel syndrome may include abdominal pain accompanied by altered bowel function, hypersensitivity, hyperalgesia, a sense of distension, intra-luminal bleeding, and flatulence. The altered bowel function may include decreased or increased frequency of bowel movements. That is, IBS may be diarrhea-predominant (D-IBS), constipation-predominant (C-IBS), or an alternating combination of both types. There is no consensus as to the cause of IBS, which appears to result from faulty regulation in both the gastrointestinal and nervous systems. That is, while the symptoms of IBS may have a physiological basis, no physiological mechanism unique to IBS has been identified. Rather, the same mechanisms that cause occasional abdominal discomfort in healthy individuals operate to produce the symptoms of IBS. The symptoms of IBS may be a product of quantitative differences in the motor reactivity of the intestinal tract, and increased sensitivity to stimuli or spontaneous contractions. Because there are no readily identifiable structural or biochemical abnormalities that result in IBS, the Rome criteria were developed to aid in diagnosis of IBS. The Rome criteria form a consensus definition of IBS. According to the Rome criteria, IBS is indicated by abdominal pain or discomfort which is (1) relieved by defecation and/or (2) associated with a change in frequency or consistency of stools, plus two or more of the following: altered stool frequency, altered stool form, altered stool passage, passage of mucus, and bloating or feeling of abdominal distention.

The walls of the gastrointestinal (GI) tract have four layers: the mucosa, submucosa, muscluaris externa, and serosa. The mucosa consists of an epithelium with basement membrane (called the lamina propria), loose connective tissue, blood vessels, and lymph tissues. The submucosa contains loose connective tissue, glands, nerves, and blood vessels. The nerve fibers of the submucosa form a network or plexus called the plexus of Meissner. The muscularis externa may include two bands of smooth muscle cells, the internal layer is composed of circular smooth muscle and the external layer is composed of longitudinal fibers. Interspersed between the muscle fibers is a nerve plexus called the plexus of Auerbach. The outermost layer of the digestive tube, the serosa, is composed of a membrane of squamous epithelium.

The gastrointestinal tract has a simplified “brain”, or nerve network, in the myenteric and submucosal plexuses, which has about 100 million neurons (the enteric nervous system). Efferents in the submucosal Meissner plexus regulate secretion by intestinal glands. The efferents in the myenteric Auerbach plexus control peristalsis, which is the rhythmic contraction of circular and longitudinal muscles. Visceral sensory afferent nerve endings are located throughout the submucosa and the Meissner plexus. Cranial nerves of the parasympathetic nervous system (e.g., the vagus) convey much of the sensory information from the gastrointestinal tract. However, sympathetic afferent nerves may transmit visceral sensations of pain to the spinal cord. Sensations, motility, digestion and secretion may be controlled by nerve elements of the gastrointestinal tract. The gastrointestinal tract submucosa contains sensory afferent nerve fibers that code for pressure, temperature and pain signals. After injury to the gastrointestinal tract, especially to the mucosa, inflammation may disrupt the enteric nervous system and contribute to gastrointestinal disorders, such as irritable bowel disease.

Current treatment options for irritable bowel syndrome range from education, exercise and dietary modification to drug therapy and psychological therapy. But, there is currently no single drug, medicine or pharmacologic treatment appropriate to all, or even most, IBS sufferers. Although largely ineffective, current treatment is multi-factorial, delivered on an ad-hoc basis, and consists of stress management, diet, and drugs, in that order. The patient may be advised to reduce or eliminate controllable stress. Relaxation exercises and biofeedback may be used in an attempt to alter the psychogenic components of the illness.

With respect to diet, the patient avoids foods to which they possess a known sensitivity with respect to exacerbating the problem. A high fiber diet, either insoluble wheat bran or soluble psyllium, is almost routinely recommended, but with little if any positive benefit. For constipation-predominant irritable bowel syndrome, suitable gastrointestinal stimulants, or transit time reducers include wheat bran, soluble fiber, and polycarbophil calcium. But, while the gastrointestinal stimulants are useful to reduce gastrointestinal transit time, they also may exacerbate abdominal pain and bloating, and exacerbate D-type irritable bowel syndrome symptoms.

With reference to drugs or medicines for the treatment of irritable bowel syndrome, none has demonstrated sufficient efficacy to be of practical benefit to a majority of patients. Drugs for the treatment of IBS either are treatments directed to the gastrointestinal tract, or treatments directed to affective disorders mediated by the central nervous system (CNS) which are associated with IBS.

Drug treatment directed to the gastrointestinal tract includes antacids, anti-spasmodic agents, anti-diarrheal drugs, anti-inflammatory drugs such as glucocorticosteroids and NSAIDS, histamine-R2-blocking agents, antibiotics, and, in the case of ulcerative colitis, surgery to remove the affected tissues. Suitable antacids include buffered acid neutralizing agents and proton pump inhibitors. Cholestyramine is a copolymer of styrene and divinylbenzene possessing trimethylbenzyl ammonium groups, and has a somewhat limited capacity to bind bile acids.

Anti-spasmodic (anti-cholinergic) medication is recommended for IBS pain and bloating. Drugs having spasmolytic activity may be prescribed to decrease intestinal motility. U.S. Pat. Nos. 4,611,011, 4,701,457, and 4,745,131 disclose a series of amidinoureas which reduce intestinal motility and are useful for treating irritable bowel syndrome. 1-Azabicyclo [2-2-2] octan-3-yl-2-aryl-3-azacyclo-2-hydroxy propionates and their quaternary salts, which possess antispasmodic activity and are useful for treating irritable bowel syndrome, are disclosed in U.S. Pat. No. 4,843,074. Calcium channel antagonists exhibit muscle relaxing and antispasmodic activities. A series of substituted imidazolyl-alkyl-piperazine and diazepine derivatives, disclosed in U.S. Pat. No. 5,043,447, are calcium channel antagonists and may be useful as antispasmodics for treating irritable bowel syndrome. U.S. Pat. No. 4,877,779 discloses 2-Aminomethylalkynylalkyl-1,3-dithiane derivatives with calcium-channel blocking activity and potentially similar uses. Some triazinone derivatives having spasmolytic activity for treating irritable bowel syndrome are disclosed in U.S. Pat. No. 4,562,188.

In addition to antispasmodic agents, compounds with other activities have been disclosed which may relieve the symptoms of irritable bowel syndrome. Anti-diarrheal agents, such as loperamide, diphenoxylate, and codeine phosphate, have been used to treat D-IBS. Unfortunately, such agents may exacerbate the constipatory phase of the disease and are ineffective in treating the additional symptoms associated with IBS, such as abdominal pain. They are, therefore, of little practical long-term benefit. Other anti-diarrheals include anti-cholinergics and smooth muscle relaxants, such as cimetropium bromide, pinaverium bromide, octilium bromide, trimebutine, and mebeverine.

U.S. Pat. No. 4,239,768 discloses a series of arylimidazolidinylidene ureas which decrease the sensitivity of the bowel to distension and thereby reduce irritable bowel symptoms. U.S. Pat. No. 4,970,207 discloses some benzodiazepine derivatives which are cholecystokinin antagonists, and which may be useful for the treatment of irritable bowel syndrome. Anti-spasmodics, anti-diarrheal preparations, analgesics and the like have been used, but even if they are effective, long-term treatment is precluded by problems such as development of tolerance, toxicity, or abuse potential.

Drugs designed to treat affective disorders mediated by the CNS include psychoactive drugs, such as some anxiolytics and some antidepressants. Even if effective for a given patient, psychoactive drugs are consider to have limited and short-term utility because of the high potential for addiction to and abuse of these agents.

Non-selective excitatory opioid receptor antagonists have been identified as central nervous system treatments that affect the symptoms associated with irritable bowel syndrome. Non-selective excitatory opioid receptor antagonists include tricyclic antidepressants, such as amitriptyline, imipramine, and doxepin, and have been used to treat irritable bowel syndrome. These opioid receptor antagonist may be effective due to the neuromodulatory and analgesic properties of these compounds, which are independent of their psychotropic effects. The non-selective nature of the tricyclic antidepressants results in affectation of all five of the recognized muscarinic receptors and may cause undesirable side effects.

Manufacturer's recommended dosages of imipramine pamoate may be modified as necessary by response and evidence of intolerance. The manufacturer's recommended dosages include initial adult dosage for outpatients starting at 75 mg/day, which may be increased to 150 mg/day—the level at which the optimum response is usually obtained for anti-depression treatment. Also for anti-depression treatment, manufacturer's recommended dosages for hospitalized patients are to start at an even higher dose of 100-150 mg/day—and the dosage can be raised as high as 300 mg/day. Elderly patients and children are stated to likely respond to a dosage of 25-50 mg/day.

Selective excitatory opioid receptor antagonist naturally have been studied in an attempt to decrease or eliminate the undesirable side effects caused by the non-selective nature of the above non-selective excitatory opioid receptor antagonist. For example, nalmefene glucuronide has been used as a treatment for constipation-predominant IBS (C-IBS). Patients receiving the composition reported a decreased transit time and increased stool frequency. Unfortunately, nalmefene glucuronide did not reduce abdominal pain or bloating, and stool consistency was not improved.

U.S. Pat. No. 5,512,578 discloses co-administration of a selective excitatory opioid receptor antagonist with a bimodally-acting opioid agonist may enhance analgesic potency, and reduce tolerance and dependence liability. Such selective excitatory opioid receptor antagonists include, when administered at appropriately low doses, naloxone, naltrexone, etorphine, and dihydroetorphine. The selective excitatory opioid receptor antagonists attenuate excitatory, but not inhibitory, opioid receptor functions in nociceptive (pain) pathways of the peripheral and central nervous systems. As a result, symptoms associated with activation of excitatory opioid receptors, such as anti-analgesia, hyperalgesia, hyperexcitability, physical dependence and/or tolerance effects may be increased.

U.S. Pat. No. 6,664,270 discloses a method and composition for treating irritable bowel syndrome using a polyamine material. Unfortunately, the composition related to diarrhea-predominant irritable bowel syndrome (D-IBS), had undesirable side effects, and was not sufficiently efficacious in the treatment.

In spite of the many treatments, compositions and methods used to reduce or eliminate symptoms associated with gastrointestinal disorders, such as irritable bowel syndrome, no suitable long-term efficacious treatment or preventative has been identified. It would be desirable to have a medicinal composition or medicine having improved properties for the treatment of irritable bowel syndrome. It also would be desirable to have a method for the treatment of irritable bowel syndrome.

### SUMMARY

The present invention relates to a method and a medicine for treating a human having a gastrointestinal disorder including irritable bowel syndrome. The medicine includes a tricyclic antidepressant and a stool softener. The method includes administering a dose of the medicine to the human.

In an aspect of the invention, invention relates to a process that includes interacting with muscarinic receptors in the human to reduce or eliminate at least one symptom caused by or associated with chronic irritable bowel system. The process further includes emulsifying oil and water into fecal matter using the surfactant to soften the stool of the human, lubricating the fecal matter to facilitate passage of the stool, or both emulsifying and lubricating the fecal matter to both soften the stool and facilitate passage of the stool.

### BRIEF DESCRIPTION OF THE DRAWINGS

FIG. 1 is a schematic diagram showing a packaging configuration of a medicine comprising an embodiment in accordance with the invention; and

FIG. 2 is a schematic block diagram showing a method in accordance with the invention.

### DESCRIPTION OF PREFERRED EMBODIMENTS

The present invention generally relates to a method of treating a disorder of the gastrointestinal (GI) tract with a medicinal composition, and the medicine. As used herein, a chronic condition refers to a condition that lasts for a substantial period or long time, and in some instances a chronic condition may not have an endpoint. Furthermore, chronic conditions may be continuous or recurring, and may reoccur regularly or irregularly. Gastrointestinal tract disorder includes irritable bowel syndrome. Unless specified or the context dictates otherwise, irritable bowel syndrome includes constipation-type irritable bowel syndrome (C-IBS), diarrhea-type irritable bowel syndrome (D-IBS), and alternating C-IBS and D-IBS. Constipation is intended to broadly include a reduced frequency of bowel movements up to and including obstipation. Diarrhea is intended to broadly include both a medical practitioner's definition—an increased frequency of bowel movements, and a lay person's definition—liquid or fluid stool that causes difficulty of continence. A medicinal composition (“medicine”) is a substance administered in the treatment of disease; a remedial agent; and/or a remedy. An efficacious amount is an amount greater than zero that has a desired or desirable effect.

A method according to embodiments of the invention includes administering a dose of the medicine to a patient suffering from or presenting symptoms associated with a gastrointestinal disorder, such as irritable bowel syndrome. The medicine is described below, as is dosage information and packaging. The irritable bowel syndrome may be a result of, for example, one or more of a nerve injury, a course of radiation treatments, a hemorrhoid surgery, a chemotherapy treatment, a compromised vascular supply to the bowel, malnutrition, diabetes, cancer, or other, possibly unknown, sources. The nerve injury may be, for example, a spinal nerve injury, spinal cord injury, or pelvic nerve injury. The compromised vascular supply to the bowel may be a result of, for example, cigarette smoking, a high cholesterol condition, collagen vascular disease, or a stroke of the bowel mesenteric artery.

In one embodiment, the medicine includes a tricyclic antidepressant and one or both of a stool softener and a fecal lubricant. Tricyclic antidepressants may be used alone or in combination and may include amitriptyline, clomipramine, desipramine, imipramine, doxepin, and nortriptyline, and derivatives and pharmaceutically acceptable salts thereof. Unless otherwise specified or indicated by context, “stool softener” will herein collectively include both stool softener and fecal lubricant for ease of referral. The medicine according to embodiments of the present invention may be used to treat C-IBS, D-IBS, and alternating type irritable bowel syndrome. The tricyclic antidepressant (that may reduce stool frequency) is present regardless of infrequent stools, and the stool softener (that may increase stool frequency) is present regardless of frequent stools.

In one embodiment, the tricyclic antidepressant includes imipramine (5-[3-(dimethylamino)propyl]-10,11-dihydro-5H-dibenzazepine), which is shown structurally below, or an active metabolite thereof—such as desmethylimipramine.

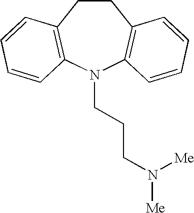

In another embodiment, the tricyclic antidepressant includes imipramine HCl. Imipramine hydrochloride is available for commercial sale as TOFRANIL from Mallinckrodt Inc. (St. Louis, Mo.). As used throughout, reference to dosage of imipramine generally will be to an equivalent amount of imipramine HCl. Also, unless specified, dosage values are in units of milligrams. For anti-depression treatment, TOFRANIL is supplied in 10, 25, 50 and 75 mg tablets or capsules.

In yet another embodiment, the tricyclic antidepressant includes imipramine pamoate (5-[3-(dimethylamino) propyl]-10,11-dihydro-5H-dibenzazepine 4,4-methlyenebis-(3-hydroxyl-2-napthoate) (2:1 ratio of pamoate to imipramine). Imipramine pamoate is commercially available as TOFRANIL-PM from Mallinckrodt Inc. in capsules of 75 mg, 100 mg, 125 mg and 150 mg dosages.

The total daily dosages of imipramine in a medicine according to the present invention are in a range of from about 10 mg/day to about 100 mg/day, about 25 mg/day to about 75 mg/day, or about 25 mg/day to about 50 mg/day. Here and elsewhere, range limitations may be combined.

Alternatively, in one embodiment the total daily dosage may be based on patient weight. According to an embodiment of the present invention a total daily dosage of imipramine in a medicine may be in a range of from about 0.1 milligram/kilogram body weight/day (mg/kg/day) to about 2.5 mg/kg/day, about 0.2 mg/kg/day to about 1.2 mg/kg/day, about 0.5 mg/kg/day to about 2.0 mg/kg/day, about 0.5 mg/kg/day to about 0.75 mg/kg/day, about 0.75 mg/kg/day to about 1.25 mg/kg/day, or about 1.25 mg/kg/day to about 2.0 mg/kg/day.

Children may have a higher glomerular filtration rate (GFR) relative to adults, and therefore the dosage may need to be adjusted upward to accommodate the higher rate, rather than downward as seen in anti-depression treatment. In one embodiment, a child dosage may be up four times greater than the adult dosages, or up to about 300 mg/day. For elderly, infirm, or smaller than average-sized patients a total daily dosage amount may be adjusted downward, for example, in a range of from about 5 mg to about 30 mg, or from about 5 mg to about 10 mg.

The daily dose(s) may be taken once a day or over the course of the day. For example, a 75 mg/day dose may be taken as 25 mg three times a day, optionally each with a meal. The tricyclic antidepressant may be taken concomitant with the stool softener or may be taken at a time different than the stool softener depending on the form, packaging and configuration of the tricyclic antidepressant and the stool softener. The regimen for taking the medicine, or components or portions thereof, is discussed further below.

Relatively decreased dosages of, for example, imipramine pamoate having a comparable effect as higher dosages may be achieved by concurrently ingesting metabolism inhibiting compositions, such as methylphenidate HCl (which is commercially available from Ciba-Geigy Corporation (Basel, Switzerland), a division of Novartis Pharmaceuticals Corporation, as RITALIN and RITALIN SR).

Stool softener as used herein is distinguished from laxatives. Laxatives may include bulk, osmotic and stimulant-type. Bulk laxatives may include soluble and insoluble fiber. Soluble fiber may include psyllium husks and is commercially available as METAMUCIL from Procter & Gamble Inc. (Cincinnati, Ohio). Insoluble fiber can include wheat bran. Osmotic laxatives are not absorbed and function by pulling water into the colon via osmotic action (e.g., magnesium hydroxide, such as PHILLIP'S MILK OF MAGNESIA, which is commercially available from Bayer Corporation (Pittsburgh, Pa.)). Stimulant laxatives interfere with absorption of water from the colon lumen and motility of fecal material therethrough.

By way of contrast, a stool softener acts to emulsify water and/or oil into fecal matter and thus soften the consistency. A fecal lubricant acts by lubricating the fecal matter and allowing it to pass though the colon with a reduced amount of friction. Suitable stool softeners include surfactants, such as anionic surfactants. Other suitable surfactants include nonionic surfactants, cationic surfactants, and amphoteric surfactants. In one embodiment, the stool softener includes bis (2-ethylhexyl) sulfosuccinate sodium salt (“docusate sodium”), which is commercially available from Purdue Phama L. P. (Stamford, Conn.) as COLACE. Other suitable metal salts of sulfosuccinate may be useful, and the metal may be potassium, calcium and the like. PERICOLACE (which is a tradename for docusate plus casanthrol), sodium dodecylsulfate (SDS), sodium cholate, sodium deoxycholate (DOC), N-lauroylsarcosine sodium salt, lauryldimethylamine-oxide (LDAO), and cetyltrimethyl ammoniumbromide (CTAB) may be used in embodiments according to the invention.

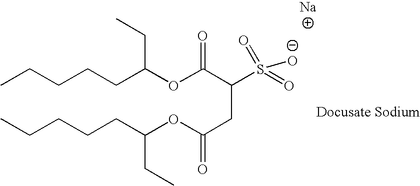

The fecal lubricant may include, for example, commercially available mineral oil or liquid paraffin. The stool softener and fecal lubricant may be used alone and in combination with each other. In combination, the stool softener can emulsify the fecal lubricant into the stool.

In one embodiment according to the invention, the stool softener may be used in efficacious amounts at dosage levels of less than 200 mg/day. In one embodiment, the dosage of stool softener may be greater than 200 mg/day, and may be used in an amount of up to about 300 mg/day, or up to about 400 mg/day. Alternatively, the amount of the stool softener may be determined with reference to body weight. In one embodiment, the total daily dosage may be in a range of from about 1 mg/kg/day to about 4 mg/kg/day. In one embodiment, the total daily dosage may be in a range of from about 1.0 mg/kg/day to about 2.0 mg/kg/day, from about 2.0 mg/kg/day to about 3.0 mg/kg/day, or from about 3.0 mg/kg/day to about 4.0 mg/kg/day.

The frequency of dosages may be determined on an individual basis. However, in one embodiment the daily dosage is 300 mg/day taken in three 100 mg doses spaced over the course of the day, optionally with a meal. As noted above the stool softener may be taken concomitant with the tricyclic antidepressant or may be taken at a different time relative to the tricyclic antidepressant. The regimen for taking the medicine, or components or portions thereof, is discussed further below.

The dosage amount of tricyclic antidepressant to stool softener may be expressed as a ratio or a proportion. In one embodiment, the ratio of tricyclic antidepressant to stool softener is in a range of from about 1:80 to about 3:1, from about 1:12 to about 1:6, from about 1:4 to about 1:3, from about 1:2 to about 1:1, or from about 2:1 to about 3:1. In one embodiment, the ratio may be preselected based on weight, symptom severity, symptom type, symptom frequency, dietary considerations, type of tricyclic antidepressant and stool softener, dose regimen, administration method, environmental considerations, other or additional medications, and the like. In one embodiment, the ratio may be selected based on individual responsiveness, dietary considerations, and environmental considerations, as well as side effects, aggravating conditions such as stress level, other or additional medications, and the like.

All or a portion of the medicine may be in the form of a pill, capsule, gelcap, a coated or chewable tablet, a chewable gum, an ingestible liquid admixture, transdermal patch, an inhalable powder or mist, an enema or suppository, an intravenous solution or an intramuscular injectable liquid.

Administration of the dose may include selecting an entry method or application based on the form of the medicine. For example, if imipramine is a gelcap, and sodium docusate is an ingestible liquid, the imipramine may be swallowed and the sodium docusate may be imbibed or drank. In one embodiment, imipramine and sodium docusate may be combined or commingled in a single capsule.

In one embodiment, imipramine and sodium docusate may be combined or commingled as portions contained in a plurality of capsules. The portions may be fractional amounts of the total daily dose. The plurality of capsules may be taken throughout the course of the day to distribute the medicine over the course of the day. For example, the medicine may be in the form of pills that each containing 50 mg of stool softener admixed with 5 mg of tricyclic antidepressant. The total daily dose may be 150 mg of stool softener and 15 mg of tricyclic antidepressant. Taking three doses of the portions over the course of a day would enable the total daily dosage to be achieved.

Similarly, the total daily dosage amount may be controlled by selecting portions containing fractional dosage amounts. For example, the total daily dosage amount may be adjusted from 150 mg of stool softener and 15 mg of tricyclic antidepressant per day to 300 mg of stool softener and 30 mg of tricyclic antidepressant per day. Taking six pills each containing 50 mg of stool softener admixed with 5 mg of tricyclic antidepressant would achieve the adjusted total daily dosage.

The number of fractional doses or portions taken per day may be adjusted to correspond to preselected factors. Such factors may include, for example, seasonal changes (e.g., dehydration, being more prevalent in summer months, may result in a temporary amelioration of fecal incontinence), aging, the natural course of the gastrointestinal disorder, stress inducing situations, and others that may affect the occurrence or severity of symptoms of the gastrointestinal disorder.

For pills, capsules, gelcaps, tablets, and the like, suitable packaging includes multi-dose packages, such as a blister pack. The blister packs may contain dosages of the medicine according to the present invention.

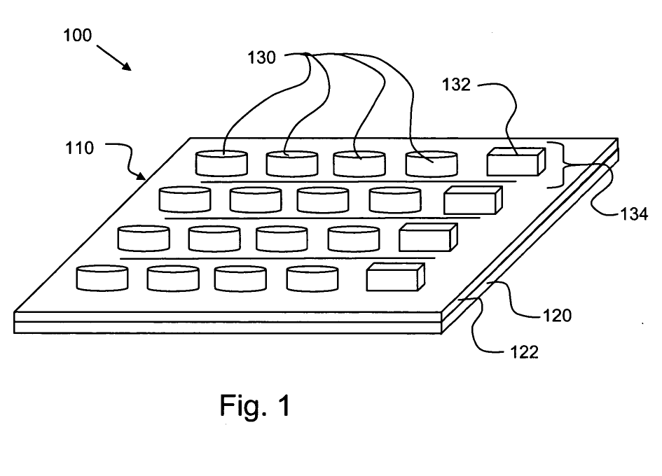

With reference to FIG. 1, a packaged treatment regimen 100 showing an embodiment according to the invention includes a blister pack 110. The blister pack 110 has a base layer 120 secured to a bottom surface of a top layer 122. The top layer 122 defines storage blisters, and the base layer 120 can operate to seal the blisters to releasably contain doses of the medicine, or portions of the medicine.

The blisters in the illustrated embodiment define differing shapes merely for the purpose of ease of differentiation. In the embodiment shown, the stool softener may be housed in the blisters labeled 130, and the tricyclic antidepressant is housed in the blister labeled 132. A row or strip 134 may equal a total daily dose of the medicine.

Because in the illustrated embodiment, the total daily dose includes four portions of stool softener (at, for example, 75 mg each) and one portion of tricyclic antidepressant (at, for example, 25 mg), there are correspondingly four blisters 130 for housing the stool softener and one blister 132 for housing the tricyclic antidepressant. Thus, the stool softener may be taken four times a day for 300 mg/day total daily dose, and the tricyclic antidepressant may be taken once a day for 25 mg/day total daily dose. Furthermore, the tricyclic antidepressant may be taken with any one of the stool softener doses, or at another time as desired.

The strip 134 is one of four shown on the blister package 110, indicating a four day supply of medicine. The blister package 110 may have instructions printed thereon that information regarding the dosage regimen, and optionally and/or additionally may include directions for varying portion dosage with reference to symptomology or exacerbating conditions.

Naturally, in other embodiments (not shown) the tricyclic antidepressant and stool softener portions may include dosages having differing amounts for different total daily dosages, may have differing numbers of doses for the same or different total daily dosages, and may have doses that include both the tricyclic antidepressant and the stool softener in a single form (such as a pill containing both the tricyclic antidepressant and the stool softener).

Other embodiments according to the invention may have the tricyclic antidepressant and/or the stool softener in a form other than pill, gel cap, and the like, and may not be amenable to blister packaging. Suitable packaging may then be selected based on the form of the tricyclic antidepressant and the stool softener, and whether the tricyclic antidepressant and the stool softener are admixed or physically separate.

With reference to forms of the medicine other than those discussed above, the ingestible liquid admixture may be administered in pre-measured amounts. The transdermal patch, the chewable gum, the intravenous solution, or the intramuscular injectable liquid, and the oral and/or nasal inhaler (for the inhalable powder or mist) may be used to deliver the tricyclic antidepressant, while the stool softener may be administered via a different method. The enema or suppository may contain the stool softener and may be administered in a conventional manner. For orally administrable embodiments in which at least one component or portion of the medicine is taken orally, masking agents may be used. For example, edible carriers, such as food, may be used to enhance palatability of the medicine or medicine component. In one embodiment, the food is selected to have a pharmacological effect. For example, prune juice has a known tendency to increase bowel movement frequency, and this tendency may be factored into the dosage amounts for the medicine or medicine components.

In one embodiment, the medicine may contain additional material either admixed or separate from the tricyclic antidepressant, the stool softener, or both. For example, the medicine may contain a skeletal muscle relaxant, a narcotic, or a proton pump inhibitor, and may further include a suitable pharmaceutical excipient, diluent, or carrier selected with regard to the intended route of administration and standard pharmaceutical practice. Suitable skeletal muscle relaxants include cyclobenzaprine hydrochloride, which may be classified as a tricyclic antidepressant and and is commercially available from McNeil Corporation (Fort Washington, Pa.) as FLEXERIL. Cyclobenzaprine hydrochloride may be combined in the medicine according to the invention. A useful dose of cyclobenzaprine hydrochloride may be 10 milligrams 4 times a day. A dosage upper limit may be about 40 milligrams a day.

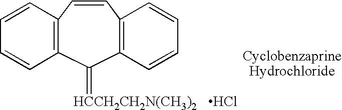

Suitable narcotics include opioid agonists include PERCOCET (oxycondone plus acetaminophen), which is commercially available from Endo Laboratories, Inc. (Chadds Ford, Pa.). Suitable proton pump inhibitors include omeprazole or 5-methoxy-2-[[(4-methoxy-3,5-dimethyl-2-pyridinyl) methyl] sulfinyl]-1H-benzimidazole, which is commercially available from AstraZeneca LP (Wilmington, Del.) as PRILOSEC, and lansoprazole, which is commercially available from TAP Pharmaceutical Products Inc. (Lake Forrest, Ill.) as PREVACID.

In one embodiment, the medicine further includes a beta-blocker, such as atenolol, which is commercially available from Medley Pharmaceuticals, Ltd (Maharashtra, India) as TENORMIN. Atenolol is a synthetic, beta1-selective (cardioselective) adrenoreceptor blocking agent or “beta-blocker”, that may be chemically described as benzeneacetamide, 4-[2′-hydroxy-3′-[(1-methylethyl) amino] propoxy] benzeneacetamide. Atenolol may block the action of the sympathetic nervous system. Because the sympathetic nervous system controls or influences the pace of the heart beat, blocking the action of these nerves can reduce the heart rate. Atenolol may reduce the force of heart muscle contraction, lower blood pressure, and may affect symptoms associated with irritable bowel syndrome, such as bowel frequency. Where tachycardia may be caused, for example, as a result of the action of the tricyclic antidepressant, a beta-blocker such as atenolol may be used to maintain the heart rate in a desired range.

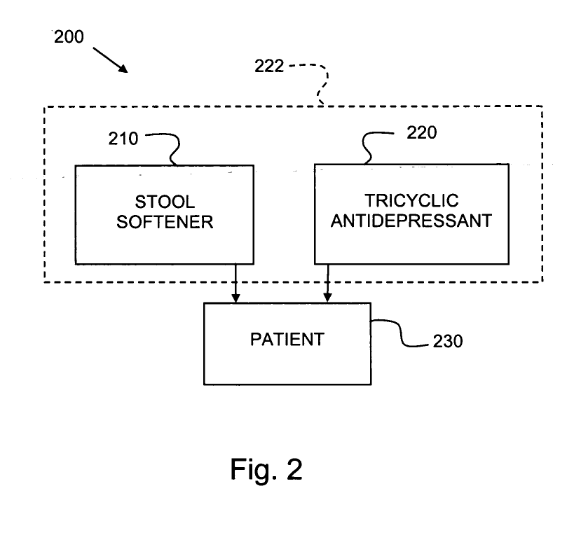

With reference to FIG. 2, a method according to the present invention is shown as a block diagram 200. A stool softener 210 and a tricyclic antidepressant 220 comprise a medicine 222. The stool softener 210 and the tricyclic antidepressant 220 are administered to a patient 230 suffering from a gastrointestinal disorder.

### EXAMPLES

Embodiments according to the invention are illustrated in the following examples. More particularly, the treatment of irritable bowel syndrome by methods and with medicines according to the present invention is shown.

### Example 1

A Caucasian female patient, 33 years old, presents with workplace injuries of sprain cervical spine and sprain lumbar spine. The spinal injuries relate to neck and back pain, with spasms, fecal urgency, irritable bowel syndrome, urinary urgency, and urinary incontinence. On presentment, protective pads are worn to absorb urine and feces.

The patient is treated with a daily dose of medicine, which includes 75 mg of imipramine pamoate and a stool softener. After several days of daily treatment via oral administration, the patient notes control of fecal urgency and irritable bowel syndrome. The patient is able to stop using protective absorbent pads during treatment. The patient reports no adverse side affects, and more particularly denies dry mouth and dry eyes.

### Example 2

A Caucasian female patient, 28 years old, presents with sprain lumbar spine. The spinal injury relates to back pain, with spasms, stress urinary incontinence and stress bowel incontinence. For example, a sneeze may result in dual bladder and bowel incontinence. On presentment, protective pads are worn to absorb urine and feces.

The patient is treated with a daily dose of medicine, which includes 75 mg of imipramine pamoate and a stool softener. After several days of daily treatment via oral administration, the patient notes control of fecal urgency and irritable bowel syndrome. The patient stops using protective absorbent pads. The patient tolerates a dry mouth.

### Example 3

A Caucasian female patient, 43 years old, presents with sprain lumbar spine. The spinal injury relates to back pain, with spasms, urgency and incontinence of the bladder and bowel.

The patient is treated with a daily dose of medicine, which includes 75 mg of imipramine pamoate. After several days of daily treatment via oral administration, the patient notes full control of bladder and bowel functions. The patient develops constipation and is prescribed docusate sodium (COLACE) in conjunction with the imipramine pamoate. The constipation is relieved by the docusate sodium.

The patient stops taking the daily dosages. After about three days without treatment, the bowel and bladder urgency and incontinence recur.

### Example 4

A Caucasian male patient, 45 years old, presents with sprain of sacrum, lumbar disc displacement, sprain lumbosacral, recurrent depression (psych-severe), and gastritis. The complaints include pain, spasms, depression, upset stomach, irritable bowel syndrome, and fecal incontinence (awake and sleeping).

The patient is treated with a total daily dose of medicine, which includes 75 mg of imipramine hydrochloride (25 mg/3 times daily) and a stool softener. After several days of daily treatment via oral administration, the patient notes partial to full control of bladder and bowel functions (awake and sleeping) and a reduction or elimination of irritable bowel syndrome symptoms. The patient tolerates a dry mouth. The patient switches to 75 mg/day (1 dosage/day) of imipramine pamoate, and a stool softener, with continued partial to full control of bowel functions (awake and sleeping) and a reduction or elimination of irritable bowel syndrome symptoms.

### Example 5

A male patient presents with a nerve injury to the spine. The complaints include irritable bowel syndrome (awake and sleeping).

The patient is treated with a total daily dose of medicine, which includes 25 mg of tricyclic antidepressant (imipramine hydrochloride) and 300 mg of stool softener (docusate sodium). After several days of daily treatment via oral administration, the patient notes partial to full control of bowel functions (awake and sleeping). That is, a reduction or elimination of irritable bowel syndrome symptoms.

### Example 6

A female patient presents with a pelvic nerve injury. The complaints include chronic, intermittent D-type irritable bowel syndrome.

The patient is treated with a total daily dose of medicine, which includes 25 mg of imipramine hydrochloride and 300 mg of stool softener (docusate sodium), ingested separately. After several days of daily treatment via oral administration, the patient notes partial to full control of bowel functions (awake and sleeping). That is, a reduction or elimination of D-type irritable bowel syndrome.

### Example 7

A male patient presents with a compromised vascular supply to the bowel. The complaints include chronic, intermittent C-type irritable bowel syndrome.

The patient is treated with a total daily dose of medicine, which includes an admixture of 5 mg of imipramine hydrochloride and 250 mg of stool softener (docusate sodium). After several days of daily treatment via oral administration, the patient notes partial to full control of bowel functions (awake and sleeping). That is, a reduction or elimination of C-type irritable bowel syndrome symptoms. A selective beta-blocker is administered in response to tachycardia on an as-needed basis.

The processes and embodiments described herein are examples of compositions, systems and methods having elements corresponding to the elements of the invention recited in the claims. This written description may enable those skilled in the art to make and use embodiments having alternative elements that likewise correspond to the elements of the invention recited in the claims. The intended scope of the invention thus includes other compositions, systems and methods that do not differ from the literal language of the claims, and further includes other compositions, systems and methods that are equivalent to, or have insubstantial differences from, the literal language of the claims.

1. A method of treating a human having a gastrointestinal disorder comprising irritable bowel syndrome, the method comprising:
administering a dose of a medicine to the human having the gastrointestinal disorder, the medicine comprising
a tricyclic antidepressant, and
a stool softener.

2. The method as defined in claim 1, wherein the gastrointestinal disorder comprises constipation type irritable bowel syndrome.

3. The method as defined in claim 1, wherein the gastrointestinal disorder comprises diarrhea type irritable bowel syndrome.

4. The method as defined in claim 1, wherein the gastrointestinal disorder comprises alternating constipation type and diarrhea type irritable bowel syndrome.

5. The method as defined in claim 1, wherein the gastrointestinal disorder is a result of one or more of a nerve injury, a course of radiation treatments, a hemorrhoid surgery, a chemotherapy treatment, or a compromised vascular supply to the bowel.

6. The method as defined in claim 5, wherein the nerve injury is a spinal nerve injury, spinal cord injury, or pelvic nerve injury.

7. The method as defined in claim 1, wherein the tricyclic antidepressant is present in an efficacious amount in a range of less than about 125 milligrams per day.

8. The method as defined in claim 7, wherein the tricyclic antidepressant is present in an amount in a range of less than about 75 milligrams per day.

9. The method as defined in claim 1, wherein the stool softener is present in an amount in a range of greater than about 200 milligrams per day.

10. The method as defined in claim 9, wherein the stool softener is present in an amount in a range of greater than about 300 milligrams per day.

11. The method as defined in claim 1, wherein the tricyclic antidepressant comprises imipramine hydrochloride, imipramine pamoate, a pharmacologically acceptable salt of imipramine, or combinations of two or more thereof.

12. The method as defined in claim 1, wherein the human is a non-elderly adult human.

13. The method as defined in claim 1, wherein the human is not clinically depressed.

14. The method as defined in claim 1, wherein the stool softener comprises a surfactant, a fecal lubricant, or a combination of surfactant and fecal lubricant.

15. The method as defined in claim 14, wherein the surfactant comprises an anionic surfactant.

16. The method as defined in claim 15, wherein the anionic surfactant comprises docusate sodium.

17. The method as defined in claim 1, wherein the gastrointestinal disorder is a chronic condition, and further comprising administering over an extended period of time corresponding to a treatment of the chronic condition.

18. The method as defined in claim 1, further comprising disposing the tricyclic antidepressant and the stool softener adjacent to each other or spaced from each other, or admixing with each other, during packaging.

19. The method as defined in claim 1, further comprising forming at least a portion of the medicine as a pill, capsule, gelcap, an ingestible liquid admixture, transdermal patch, an inhalable powder or mist, an enema or suppository, a coated or chewable tablet, a chewable gum, an intravenous solution, or an intramuscular injectable liquid, and administering comprises selecting an entry method into the human based on the form of the medicine.

20. The method as defined in claim 1, wherein the tricyclic antidepressant is administered in an efficacious amount in a range of about 0.1 mg/kg/day to about 2.5 mg/kg/day.

21. The method as defined in claim 20, wherein the tricyclic antidepressant is administered in an efficacious amount in a range of about 0.5 mg/kg/day to about 2 mg/kg/day.

22. The method as defined in claim 1, wherein the tricyclic antidepressant to stool softener ratio is in a range of from about 1:80 to about 3:1.

23. The method as defined in claim 22, wherein the tricyclic antidepressant to stool softener ratio is in a range of from about 1:4 to about 1:3.

24. The method as defined in claim 1, wherein the stool softener is administered in an efficacious amount in a range of about 1 mg/kg/day to about 4 mg/kg/day.

25. The method as defined in claim 24, wherein the stool softener is administered in an efficacious amount in a range of about 2 mg/kg/day to about 3 mg/kg/day.

26. The method as defined in claim 1, further comprising administering the tricyclic antidepressant and the stool softener is substantially simultaneous or sequentially relative to each other.

27. The method as defined in claim 1, further comprising varying amounts of the tricyclic antidepressant and the stool softener over a course of treatment in response to preselected factors.

28. The method as defined in claim 1, further comprising administering to the human a beta-blocker that is responsive to reduce tachycardia, a skeletal muscle relaxant, a narcotic, a proton pump inhibitor, or two or more thereof.

29. A medicinal composition for treating a human having a gastrointestinal disorder comprising irritable bowel syndrome, the composition comprising:
a tricyclic antidepressant, and
a stool softener.

30. The composition as defined in claim 29, wherein the irritable bowel syndrome is constipation type.

31. The composition as defined in claim 29, wherein the irritable bowel syndrome is diarrhea type.

32. The composition as defined in claim 29, wherein the irritable bowel syndrome is alternating constipation type and diarrhea type.

33. The composition as defined in claim 29, wherein the gastrointestinal disorder is a result of one or more of a nerve injury, a course of radiation treatments, a hemorrhoid surgery, a chemotherapy treatment, or a compromised vascular supply to the bowel.

34. The composition as defined in claim 33, wherein the nerve injury is a spinal nerve injury, spinal cord injury, or a pelvic nerve injury.

35. The composition as defined in claim 29, wherein the tricyclic antidepressant is present in an efficacious amount in a range of less than about 125 milligrams per total daily dose.

36. The composition as defined in claim 35, wherein the tricyclic antidepressant is present in an efficacious amount in a range of less than about 75 milligrams per total daily dose.

37. The composition as defined in claim 29, wherein the stool softener is present in an amount in a range of greater than about 200 milligrams per total daily dose.

38. The composition as defined in claim 37, wherein the stool softener is present in an amount in a range of greater than about 300 milligrams per total daily dose.

39. The composition as defined in claim 29, wherein the tricyclic antidepressant comprises imipramine hydrochloride, imipramine pamoate, a pharmacologically acceptable salt of imipramine, or combinations of two or more thereof.

40. The composition as defined in claim 29, wherein the human is a non-elderly adult.

41. The composition as defined in claim 29, wherein the human is not clinically depressed.

42. The composition as defined in claim 29, wherein the stool softener comprises a surfactant or a fecal lubricant, or a combination of surfactant and fecal lubricant.

43. The composition as defined in claim 42, wherein the surfactant comprises docusate sodium.

44. The composition as defined in claim 29, further comprising a beta-blocker that is responsive to reduce tachycardia, a skeletal muscle relaxant, a narcotic, a proton pump inhibitor, or two or more thereof.

45. The composition as defined in claim 29, wherein medicine is configured as fractional dosage amounts, the fractional dosage amounts being operable to effect variations in a total daily dosage amount of the tricyclic antidepressant and of the stool softener over a course of treatment.

46. The composition as defined in claim 29, wherein the tricyclic antidepressant and the stool softener in the medicine are configured for packaging to be adjacent to each other in each dose or admixed with each other in each dose for administration substantially simultaneously with each other; or
the tricyclic antidepressant and the stool softener in the medicine are packaged separate from each other for administration substantially simultaneously, sequentially, or alternating periodically with each other.

47. The composition as defined in claim 29, wherein at least a portion of the medicine is in the form of a pill, capsule, gelcap, an ingestible liquid admixture, transdermal patch, an oral or nasal inhalable powder or mist, an enema or suppository, a coated or chewable tablet, a chewable gum, an intravenous solution, or an intramuscular injectable liquid.

48. The composition as defined in claim 29, further comprising packaging, wherein the medicine in the form of a plurality of co-packaged dosages of the medicine, and each dose comprises a portion of a daily dosage amount, wherein
each dose comprises the tricyclic antidepressant and the stool softener, or
a first portion of the plurality of dosages comprises the tricyclic antidepressant and not the stool softener, and a second portion of the plurality of dosages includes the stool softener and not the tricyclic antidepressant, and doses of the first portion and the second portion are administrable to form a total daily dose.

49. The composition as defined in claim 29, wherein the tricyclic antidepressant is administered in an efficacious amount in a range of about 0.1 mg/kg/day to about 2.5 mg/kg/day.

50. The composition as defined in claim 49, wherein the tricyclic antidepressant is administered in an efficacious amount in a range of about 0.5 mg/kg/day to about 2 mg/kg/day.

51. The composition as defined in claim 29, wherein the tricyclic antidepressant to stool softener ratio is in a range of from about 1:80 to about 3:1.

52. The composition as defined in claim 51, wherein the tricyclic antidepressant to stool softener ratio is in a range of from about 1:4 to about 1:3.

53. The composition as defined in claim 29, wherein the stool softener is administered in an efficacious amount in a range of about 1 mg/kg/day to about 4 mg/kg/day.

54. The composition as defined in claim 53, wherein the stool softener is administered in an efficacious amount in a range of about 2 mg/kg/day to about 3 mg/kg/day.

55. A treatment kit for a human having a gastrointestinal disorder comprising irritable bowel syndrome, the kit comprising:
a plurality of doses of a medicine, the medicine comprising:
a tricyclic antidepressant, and
a stool softener comprising one or more of a surfactant and a fecal lubricant.

56. The kit as defined in claim 55, further comprising an instruction set comprising directions for administering the medicine, the instruction set comprising dosage amounts, dosing schedules, directions for varying dosages, or combinations of two or more thereof.

57. A process for treating a gastrointestinal disorder comprising irritable bowel syndrome in a human, the process comprising:
causing interaction with muscarinic receptors in the human to reduce or eliminate the gastrointestinal disorder by affecting a stool of the human, and
delivering into fecal matter an oil and water by emulsification using a surfactant to soften the stool of the human,
delivering into fecal matter a fecal lubricant to facilitate passage of the stool, or
emulsifying and lubricating the fecal matter to both soften the stool and facilitate passage of the stool,
wherein the emulsifying, lubricating, or emulsifying and lubricating occurs in the bowel of the human.
